(19) United States
(12) Patent Application Publication
(10) Pub. No.: US 2003/0144339 A1
(43) Pub. Date: Jul. 31, 2003

(54) Indole derivatives as mcp-1 receptor antagonists

(76) Inventors: Alan Wellington Faull, Macclesfield (GB); Jason Grant Kettle, Macclesfield (GB)

Correspondence Address: MORGAN LEWIS & BOCKIUS LLP, 1111 PENNSYLVANIA AVENUE NW, WASHINGTON, DC, 20004, US

(21) Appl. No.: 10/169,717

(22) PCT Filed: Jan. 11, 2001
(86) PCT No.: PCT/GB01/00069

(30) Foreign Application Priority Data
Jan. 13, 2000 (EP) 0000626.2

(51) Int. Cl. A61K 31/405; C07D 209/12; C07D 209/18

(52) U.S. Cl. 514/419; 548/494

(57) ABSTRACT

A compound of formula (I) wherein: R¹ is hydrogen, halo or methoxy; R² is hydrogen, halo, methyl, ethyl or methoxy; R³ is a halo group or a trifluoromethyl group; R⁴ is a halo group or a trifluoromethyl group; R⁵ is hydrogen or halo; R⁶ is hydrogen or halo; provided that when R⁵ and R⁶ are both hydrogen, and one of R³ or R⁴ is chloro or fluoro, then the other is not chloro or fluoro; or a pharmaceutically acceptable salt or prodrug thereof. These compounds have useful activity for the treatment of inflammatory disease, specifically in antagonising an MCP-1 mediated effect in a warm-blooded animal such as a human being.

The present invention relates to anti-inflammatory compounds that act via antagonism of the CCR2 receptor, (also known as the MCP-1 receptor), leading inter alia to inhibition of Monocyte Chemoattractant Protein-1 (MCP-1). These compounds contain an indole moiety. The invention further relates to pharmaceutical compositions containing them, processes for their preparation, intermediates useful in their preparation and to their use as therapeutic agents.

MCP-1 is a member of the chemokine family of pro-inflammatory proteins which mediate leukocyte chemotaxis and activation. MCP-1 is a C-C chemokine which is one of the most potent and selective T-cell and monocyte chemoattractant and activating agents known. MCP-1 has been implicated in the pathophysiology of a large number of inflammatory diseases including rheumatoid arthritis, glomerular nephritides, lung fibrosis, restenosis (international Patent Application WO 94/09128), alveolitis (Jones et al., 1992, J. Immunol., 149, 2147) and asthma. Other disease areas where MCP-1 is thought to play apart in their pathology are atherosclerosis (e.g. Koch et al., 1992, J. Clin. Invest., 90, 772-779), psoriasis (Deleuran et al., 1996, J. Dermatological Science, 13,. 228-236), delayed-type hypersensitivity reactions of the skin, inflammatory bowel disease (Grimm et al., 1996, J. Leukocyte Biol., 59,. 804812), multiple sclerosis and brain trauma (Berman et al, 1996, J. Immunol., 156,. 3017-3023). An MCP-1 inhibitor may also be useful to treat stroke, reperfusion injury, ischemia, myocardial infarction and transplant rejection.

MCP-1 acts through the CCR2 receptor. MCP-2 and MCP-3 may also act, at least in part, through this receptor. Therefore in this specification, when reference is made to “inhibition or antagonism of MCP-1” or “MCP-1 mediated effects” this includes inhibition or antagonism of MCP-2 and/or MCP-3 mediated effects when MCP-2 and/or MCP-3 are acting through the CCR2 receptor.

The applicants have found a class of compounds containing an indole moiety which have useful inhibitory activity against MCP-1. International Patent Application, Publication No. WO 99/07351 discloses a class of indoles with MCP-1 inhibitory activity. This application is based on the surprising discovery that particular substituted 5-hydroxy indoles are MCP-1 inhibitors which possess unexpected and beneficial properties with respect to potency and/or blood levels and/or bioavailability and/or solubility.

Accordingly, the present invention provides a compound of the formula (I):

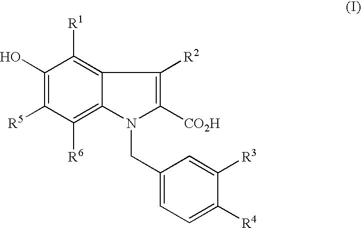

wherein:

R¹ is hydrogen, halo or methoxy,

R² is hydrogen, halo, methyl, ethyl or methoxy;

R³ is a halo group or a trifluoromethyl group;

R⁴ is a halo group or a trifluoromethyl group;

R⁵ is hydrogen or halo;

R⁶ is hydrogen or halo;

provided that when R⁵ and R⁶ are both hydrogen, and one of R³ or R⁴ is chloro or fluoro, then the other is not chloro or fluoro;

or a pharmaceutically acceptable salt or prodrug thereof.

In this specification the term “alkyl” includes both straight and branched chain alkyl groups but references to individual alkyl groups such as “propyl” are specific for the straight chain version only. The term “halo” refers to fluoro, chloro, bromo and iodo.

Suitable examples of R¹ are hydrogen, fluoro, chloro, bromo, iodo or methoxy. Preferably R¹ is hydrogen, fluoro or chloro, and most preferably R¹ is hydrogen.

Particular examples of R² are hydrogen, fluoro, chloro, bromo, iodo, methyl, ethyl or methoxy. Suitably R² is hydrogen, chloro, bromo, iodo or methoxy, and preferably R¹ is hydrogen.

In one embodiment, R⁵ and R⁶ are both hydrogen. In this case, when R⁴ is trifluoromethyl, R³ is suitably a chloro, fluoro, bromo or iodo group, preferably a chloro, fluoro or bromo group, and most preferably chloro or fluoro.

Alternatively, where R⁵ and R⁶ are both hydrogen, R³ is trifluoromethyl, and R⁴ is halo such as fluoro, chloro, bromo or iodo, and preferably chloro or fluoro and most preferably chloro.

Similar combinations of R³ and R⁴ may apply where at least one of R⁵ and 1⁶ is other than hydrogen, but in this case, R³ and R⁴ are suitably both halo such as fluoro, chloro, bromo and iodo, preferably fluoro, chloro or bromo, and most preferably fluoro or chloro. Particular examples are cases where R³ and R⁴ are both chloro, or le and 1⁴ are both fluoro. A further alternative is one in which one of R³ or R⁴ is chloro and the other is fluoro.

Suitably R⁵ is hydrogen, fluoro, chloro or bromo, and preferably R⁵ is hydrogen. A further preferred value for R⁵ is, for example, fluoro.

Suitably R⁶ is hydrogen, fluoro, chloro or bromo. Preferably R⁶ is hydrogen or fluoro, and most preferably hydrogen.

In a preferred aspect of the invention there is provided a compound of formula (IA):

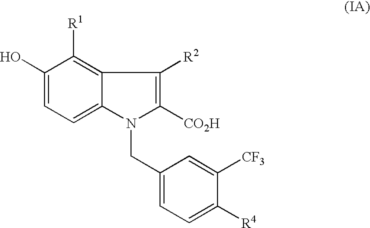

or a pharmaceutically acceptable salt or prodrug thereof, wherein R¹, R² and R⁴ are as defined above. Preferably R¹ and R² are hydrogen Preferably R⁴ is chloro or fluoro.

In a further preferred aspect of the invention there is provided a compound of formula I or a pharmaceutically acceptable salt or prodrug thereof wherein R¹, R² and R⁴ are as defined above, R³ is trifluoromethyl, R⁵ is halo and R⁶ is hydrogen Preferably le and R² are hydrogen. Preferably R⁴ is chloro or fluoro, especially chloro. Preferably R⁵ is fluoro.

Preferred compounds of the invention include any one of the compounds prepared in the Examples, which are summarised in Table 1.

TABLE 1

| Example | R1 | R2 | R3 | R4 | R5 | R6 |
|---|---|---|---|---|---|---|
| 1 | H | H | CF₃ | Cl | H | H |
| 2 | H | H | F | CF₃ | H | H |
| 3 | H | H | Cl | CF₃ | H | H |
| 4 | H | H | Br | Cl | H | H |
| 5 | H | H | F | Br | H | H |
| 6 | H | H | Br | F | H | H |
| 7 | F | H | CF₃ | F | H | H |
| 8 | F | H | CF₃ | Cl | H | H |
| 9 | F | H | CF₃ | F | F | H |
| 10 | Cl | H | Cl | Cl | Cl | H |
| 11 | H | Br | CF₃ | F | H | H |
| 12 | H | Br | CF₃ | Cl | H | H |
| 13 | H | Br | Cl | CF₃ | H | H |
| 14 | H | Cl | F | CF₃ | H | H |
| 15 | H | I | F | CF₃ | H | H |
| 16 | H | CH₃O | CF₃ | Cl | H | H |
| 17 | H | H | CF₃ | F | Cl | H |
| 18 | H | H | CF₃ | Cl | Cl | H |
| 19 | H | H | CF₃ | Cl | H | F |
| 20 | H | H | CF₃ | Cl | Br | H |
| 21 | H | H | Cl | Cl | Br | H |
| 22 | H | H | CF₃ | Cl | F | H |
| 23 | H | H | Cl | Cl | F | H |
| 24 | H | H | Cl | Cl | Cl | H |
| 25 | Cl | H | CF3 | Cl | H | H |
| 26 | Cl | H | CF3 | Cl | Cl | H |

The invention further relates to all tautomeric forms of the compounds of formula (I).

It is also to be understood that certain compounds of the formula (I) can exist in solvated as well as unsolvated forms such as, for example, hydrated forms. It is to be understood that the invention encompasses all such solvated forms.

Compounds of formula (I) are inhibitors of monocyte chemoattractant protein-1. In addition, they appear to inhibit RANTES induced chemotaxis. RANTES (Regulated upon Activation, Normal T-cell Expressed and Secreted) is another chemokine from the same family as MCP-1, with a similar biological profile, but acting though the CCR1 receptor. Accordingly a further advantage associated with the present invention is that, by inhibition of both MCP-1 and RANTES activity, it provides compounds with particularly useful properties. As a result, these compounds can be used to treat disease mediated by these agents, in particular inflammatory disease.

Suitable pharmaceutically acceptable salts of compounds of formula (I) include base salts such as an alkali metal salt for example sodium, an alkaline earth metal salt for example calcium or magnesium, an organic amine salt for example triethylamine, morpholine, N-methylpiperidine, N-ethylpiperidine, procaine, dibcnzylanine, N,N-dibenzylethylamine or amino acids for example lysine. In another aspect, where the compound is sufficiently basic, suitable salts include acid addition salts such as methanesulphonate, fumarate, hydrochloride, hydrobromide, citrate, maleate and salts formed with phosphoric and sulphuric acid. There may be more than one cation or anion depending on the number of charged functions and the valency of the cations or anions. A preferred pharmaceutically acceptable salt is a sodium salt.

Various forms of prodrugs are known in the art. For examples of such prodrug derivatives, see:

a) Design of Prodrugs, edited by H. Bundgaard, (Isevier, 1985) and Methods in Enzymology, Vol. 4, p. 309-396, edited by K. Widder, et al. (Academic Press, 1985);

b) A Textbook of Drug Design and Development, edited by Krogsgaard-Larsen and H. Bundgaard, Chapter 5 “Design and Application of Prodrugs”, by H. Bundgaard p. 113-191 (1991);

c) H. Bundgaard, Advanced Drug Delivery Reviews, 8, 1-38 (1992);

d) H. Bundgaard, et al., Journal of Pharmaceutical Sciences, 77, 285 (1988); and

e) N. Kakeya, et al., Chem Pharm Bull, 32, 692 (1984).

Examples of such prodrugs are in vivo cleavable esters of a compound of the invention. An in vivo cleavable ester of a compound of the invention containing a carboxy group is, for example, a pharmaceutically-acceptable ester which is cleaved in the human or animal body to produce the parent acid. Suitable pharmaceutically-acceptable esters for carboxy include C₁₋₆alkyl esters, for example methyl or ethyl; C₁₋₆alkoxymethyl esters, for example methoxymethyl; C₁₋₆alkanoyloxymethyl esters, for example pivaloyloxymethyl; phthalidyl esters; C₃₋₈cycloalkoxycarbonyloxyC₁₋₆alkyl esters, for example 1-cyclohexylcarbonyloxyethyl; 1,3-dioxolan-2-ylmethyl esters, for example 5-methyl-1,3-dioxolan-2-ylmethyl; C₁₋₆aLkoxycarbonyloxyethyl esters, for example 1-methoxycarbonyloxyethyl; aminocarbonylmethyl esters and mono- or di-N-(C₁₋₆alkyl) versions thereof, for example N,N-dimethylaminocarbonylmethyl esters and N-ethylaminocarbonylmethyl esters; and may be formed at any carboxy group in the compounds of this invention. An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include C₁₋₆alkanoyl esters, for example acetyl esters; and benzoyl esters wherein the phenyl group may be substituted with aminomethyl or N-substituted mono- or di-C₁₋₆allyl aminomethyl, for example 4-aminomethylbenzoyl esters and 4-N,N-dimethylaminomethylbenzoyl esters.

Further examples of such prodrugs are in vivo cleavable amides of a compound of the invention. Examples of such in vivo cleavable amides include an N-C₁₋₆alkylamide and an N,N-di-(C₁₋₆alkyl)amide such as N-methyl, N-ethyl, N-propyl, N,N-dimethyl, N-ethyl-N-methyl or N,N-diethylamide.

Another aspect of the present invention provides a process for preparing a compound of formula (I) or a pharmaceutically acceptable salt or prodrug thereof which process comprises:

a) reacting compounds of formula (II):

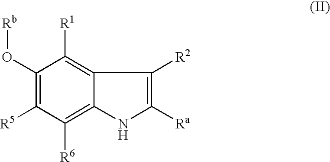

where R¹, R², R⁵ and R⁶ are as defined in relation to formula (I), R^{a} is carboxy or a protected form thereof, and R^{b} is hydrogen or a suitable hydroxy protecting group, with a compound of formula (II):

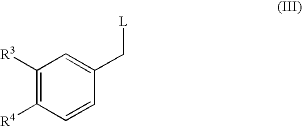

where R³ and R⁴ are as defined in relation to formula (I) and L is a displaceable group; and thereafter if necessary:

i) converting a compound of the formula (I) into another compound of the formula (I);

ii) removing any protecting groups; or

iii) forming a pharmaceutically acceptable salt or prodrug thereof.

Suitable values for L are for example, a halogeno or sulphonyloxy group, for example a chloro, bromo, methanesulphonyloxy or toluene-4-sulphonyloxy group. Compounds of formula (II) and (III) are suitably reacted together in an inert organic solvent such as N,N-dimethylformamide, dichloromethane or acetonitrile in the presence of a base such as sodium hydroxide, sodium hydride or potassium carbonate. Suitably the reaction is effected in the presence of a phase transfer catalyst such as tetra-n-butylammonium hydrogensulphate. Reaction times may range for 1-6 hours preferably for 1-3 hours. Moderate temperatures for example of 15-30° C., preferably 20-25° C. are employed.

Compounds of formula (HI) may be commercially available, or they may be made by modification using known processes of commercially available compounds of formula (a). In particular, they may be prepared by reacting a compound of formula (IV):

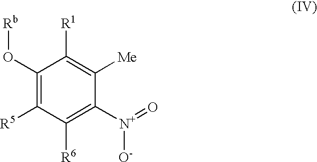

where R¹, R¹, R⁶ and R^{b} is as defined above with a compound of formula (V)

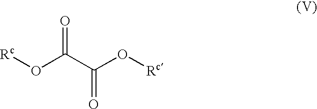

where R^{c} and R^{c}, are independently selected from C₁₋₄alkyl.

Compounds of formula (IV) and (V) are suitably reacted together under Reissert reaction conditions such as in an inert solvent (such as tetrahydrofuran), in the presence of a base (such as potassium ethoxide), at a temperature range of 15-30° C. preferably 20-25° C., for 10-20 hours preferably 15-17 hours. The resulting compound is isolated and dissolved in an alcohol such as ethanol and an organic acid (such as acetic acid) and a transition metal catalyst (such as 10% Pd/C) and cyclohexene is added. The mixture may then be heated at a temperature of 60-120° C. preferably at 70-90° C. for 15-25 hours preferably 16-20 hours to give a compound of formula ([1) wherein R^{a} is —CO₂R^{c}.

R^{c} and R^{c}, are suitably C₁₋₄alkyl, preferably methyl or ethyl.

Alternatively, compounds of formula (H) can be prepared by reacting a compound of formula (VI):

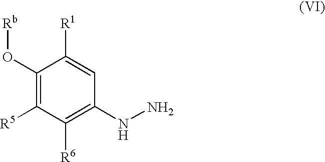

where R¹, R⁵, R⁶ and R^{b} are as defined above, with a compound of formula (VII):

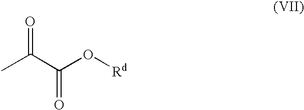

where R^{d} is C₁₋₄alkyl.

Suitably R^{d} is C₁₋₄alkyl, preferably methyl or ethyl. Compounds of formula (VI) and (VII are suitably reacted together under Fischer conditions such as with an organic acid (such as acetic acid), in an alcohol (such as ethanol), at a temperature of 60-90° C., preferably 75-85° C., for 1-S hours, preferably 1-3 hours. The resulting compound is mixed with a strong acid (such as polyphosphoric acid) and heated at 90-150° C. preferably 100-120° C., for 0.5-4 hours, preferably 0.5-2 hours to give a compound of formula (II) in which R² is hydrogen. Then, if desired, R² can be optionally converted into another value of R² as defined in formula (I) using techniques known in the literature.

In a preferred alternative, compounds of formula (II) are obtained by cyclisation of a compound of formula (VIII)

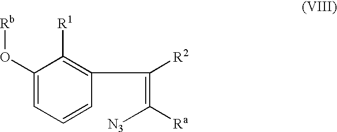

where R¹, R^{a}, R^{b} and R² are as defined above.

Cyclisation may be effected by refluxing the compound in an organic solvent such as xylene. Compounds of formula (VIII) are suitably prepared by reacting a compound of formula (IX)

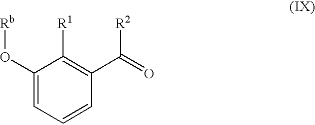

where R¹, R² and R^{b} are as defined above, with a compound of formula (X)

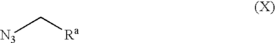

where R^{a} is as defined above. The reaction is suitably effected in an organic solvent such as an alcohol, in particular methanol, in the presence of a base such as an alkali metal alkoxide, in particular sodium methoxide. Moderate temperatures of from −30 to 20° C. are suitably employed.

In yet a further modification, compounds of formula (II) are prepared by cyclisation of a compound of formula (XI)

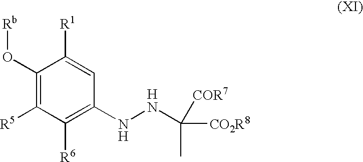

where R¹ and R^{b} are as defined above, R⁷ is alkyl, such as methyl, and R⁸ is a carboxy protecting group such as alkyl, in particular methyl.

Cyclisation is suitably effected under Japp Klingemann conditions, by warming a solution of the compound in an organic solvent such as toluene and a suitable acid, such as p-toluene sulphonic acid.

Compounds of formula (XI) are suitably prepared by reacting a compound of formula (XII)

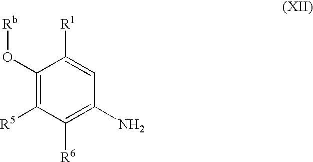

where R¹, R^{b}, R⁵ and R⁶ are as defined above, with a compound of formula (XI)

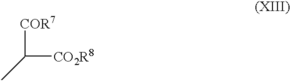

where R⁷ and R⁵ are as defined in relation to formula (XI). The compound of formula (XII) is suitably dissolved in a dilute acid such as 1.5N HCl in the presence of a nitrite such as sodium nitrite at moderately low temperatures from −30 to 0° C., preferably −5° C.

This solution is then mixed with a solution of a compound of formula (XIII) in an organic solvent such as ethanol, in the presence of a solution of a base such as an alkali metal hydroxide, for example aqueous sodium hydroxide solution.

Compounds of formula (III), (IV), (V), (VI), (VII), (IX), (X) and (XII) are known or commercially available or are prepared by processes known in the art by standard manipulation of commercially available or known materials.

It will also be appreciated that in some of the reactions mentioned herein it maybe necessary/desirable to protect any sensitive groups in the compounds. The instances where protection is necessary or desirable and suitable methods for protection are known to those skilled in the art. Thus, if reactants include groups such as carboxy or hydroxy it may be desirable to protect the group in some of the reactions mentioned herein.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esteriifing group, for example a methyl or an ethyl group which maybe removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Some of the intermediates described herein may be novel, for example intermediates of the formula (II), and as such they are provided as a further feature of the invention.

When a pharmaceutically-acceptable salt of a compound of formula (I) is required, it may be obtained, for example, by reaction of said compound with the appropriate acid (which affords a physiologically acceptable anion), or with the appropriate base (which affords a physiologically acceptable cation), or by any other conventional salt formation procedure.

According to a further aspect of the invention there is provided a pharmaceutical composition which comprises a compound of the formula (I) as defined hereinbefore or a pharmaceutically acceptable salt or prodrug thereof, in association with a pharmaceutically acceptable excipient or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention maybe obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations maybe uncoated or coated either to modify their disintegration and the subsequent absorption of the active ingredient within the gastrointestinal track, or to improve their stability and/or appearance, in either case, using conventional coating agents and procedures well known in the art.

Compositions for oral use maybe in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols. Topical formulations, such as creams, ointments, gels and aqueous or oily solutions or suspensions, may generally be obtained by formulating an active ingredient with a conventional, topically acceptable, vehicle or dilaent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergaamon Press 1990.

The size of the dose for therapeutic or prophylactic purposes of a compound of the Formula I will naturally vary according to the nature and severity of the conditions, the age and sex of the animal or patient and the route of administration, according to well known principles of medicine. As mentioned above, compounds of the Formula I are useful in treating diseases or medical conditions which are due alone or in part to the effects of MCP-1 and/or RANTES, for example, rheumatoid arthritis.

In using a compound of the Formula I for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

According to a further aspect of the present invention there is provided a compound of the formula (I) or a pharmaceutically acceptable salt or prodrug thereof, as defined hereinbefore for use in a method of treatment of the human or animal body by therapy. Conveniently, the invention provides a method of treating inflammatory disease by administering a compound of formula (I) or a pharmaceutically acceptable salt or prodrug or a pharmaceutical composition thereof, as described above.

A further feature of the present invention is a compound of formula (I) and pharmaceutically acceptable salt or prodrug thereof, for use as a medicament.

Conveniently this is a compound of formula (I), or a pharmaceutically acceptable salt or prodrug thereof, for use as a medicament for antagonising an MCP-1 mediated effect (and/or a RANTES mediated effect) in a warm-blooded animal such as a human being.

Thus according to a further aspect of the invention there is provided the use of a compound of the formula (I), or a pharmaceutically acceptable salt or prodrug thereof, in the manufacture of a medicament for use in antagonising an MCP-1 mediated effect (and/or a RANTES mediated effect) in a warm-blooded animal such as a human being.

According to a further feature of the invention there is provided a method of antagonising an MCP-1 mediated effect in a warm-blooded animal, such as a human being, in need of such treatment which comprises administering to said animal an effective amount of a compound of formula (I) or a pharmaceutically acceptable salt or prodrug thereof, as defined hereinbefore.

Biological Testing.

The following biological test methods, data and Examples serve to illustrate the present invention.

Abbreviations:

| | |
|---|---|
| ATCC | American Type Culture Collection, Rockville, USA. |
| BCA | Bicinchroninic acid, (used, with copper sulphate, |
| | to assay protein) |
| BSA | Bovine Serum Albumin |
| DMEM | Dulbecco's modified Eagle's medium |
| EGTA | Ethylenebis(oxyethylenenitrilo)tetraacetic acid |
| FCS | Foetal calf serum |
| HEPES | (N-[2-Hydroxyethyl]piperazine-N′-[2-ethanesulphonic acid]) |
| HBSS | Hank's Balanced Salt Solution |
| hMCP-1 | Human Monocyte Chemoattractant Protein-1 |
| PBS | Phosphate buffered saline |
| PCR | Polymerase chain reaction |

AMPLITAQ™, available from Perkin-Elmer Cetus, is used as the source of thermostable DNA polymerase.

Binding Buffer is 50 mM HEPES, 1 mM CaCl₂, 5 mM MgCl₂, 0.5% foetal calf serum, adjusted to pH 7.2 with 1 M NaOH.

Non-Essential Amino Acids (100× concentrate) is: L-Alanine, 890 mg/l;

L-Asparagine, 1320 mg/l; L-Aspartic acid, 1330 mg/l; L-Glutamic acid, 1470 mg/l; Glycine, 750 mg/l; L-Proline, 1150 mg/l and; L-Serine, 1050 mg/l.

Hypoxanthine and Thymidine Supplement (50×concentrate) is: hypoxanthine, 680 mg/l and; thymidine, 194 mg/L.

Penicillin-Streptomycin is: Penicillin G (sodium salt); 5000 units/ml; Streptomycin sulphate, 5000 μg/ml.

Human monocytic cell line THP-1 cells are available from ATCC, accession number ATCC TIB-202.

Hank's Balanced Salt Solution (HBSS) was obtained from Gibco; see Proc. Soc. Exp. Biol. Med., 1949,71,196.

Synthetic cell culture medium, RPMI 1640 was obtained from Gibco; it contains inorganic salts [Ca(NO₃)₂.4H₂O 100 mg/l; KCl 400 mg/l; MgSO₄.7H₂O 100 mg/l; NaCl 6000 mg/l; NaHCO₃ 2000 mg/l & Na₂PO₄ (anhyd) 800 mg/l], D-Glucose 2000 mg/l, reduced glutathione 1 mg/l, amino acids and vitamins.

FURA-2/AM is 1-[2-(5-carboxyoxazol-2-yl)-6-aminobenzofuran-5-oxy]-2-(2′-amino-5′-methylphenoxy)-ethane-N,N′,′-tetraacetic acid pentaacetoxymethyl ester and was obtained from Molecular Probes, Eugene, Oreg., USA.

Blood Sedimentation Buffer contains 8.5 g/l NaCl and 10 g/l hydroxyethyl cellulose.

Lysis Buffer is 0.15M NH₄Cl⁻, 10 mM KHCO₃, 1 mM EDTA

Whole Cell Binding Buffer is 50 mM HEPES, 1 mM CaCl₂, 5 mM MgCl₂, 0.5% BSA, 0.01% NaN₃, adjusted to pH 7.2 with 1M NaOH.

Wash buffer is 50 mM HEPES. 1 mM CaCl₂, 5 mM MgCl₂, 0.5% heat inactivated FCS, 0.5MNaCl adjusted to pH 7.2 with 1M NaOH.

General molecular biology procedures can be followed from any of the methods described in “Molecular Cloning—A Laboratory Manual” Second Edition, Sambrook, Fritsch and Maniatis (Cold Spring Harbor Laboratory, 1989).

i) Cloning and Expression of hMCP-1 Receptor

The MCP-1 receptor B (CCR2B) cDNA was cloned by PCR from ThP-1 cell RNA using suitable oligonucleotide primers based on the published MCP-1 receptor sequences (Charo et al., 1994, Proc. Natl. Acad. Sci. USA, 91, 2752). The resulting PCR products were cloned into vector PCR-II™ (In Vitrogen, San Diego, Calif.). Error free CCR2B cDNA was subcloned as a Hind III-Not I fragment into the eukaryotic expression vector pCDNA3 (In Vitrogen) to generate pCDNA3/CC-CKR2A and pCDNA3/CCR2B respectively.

Linearised pCDNA3/CCR2B DNA was transfected into CHO-K1 cells by calcium phosphate precipitation (Wigler et al., 1979, Cell, 16, 777). Transfected cells were selected by the addition of Geneticin Sulphate (G418, Gibeo BRL) at 1 mg/ml, 24 hours after the cells had been transfected. Preparation of RNA and Northern blotting were carried out as described previously (Needham et al., 1995, Prot. Express. Purific., 6, 134). CHO-K1 clone 7 (CHO-CCR2B) was identified as the highest MCP-1 receptor B expresser.

ii) Preparation of Membrane Fragments

CHO-CCR2B cells were grown in DMEM supplemented with 10% foetal calf serum, 2 mM glutamine, 1× Non-Essential Amino Acids, 1× Hypoxantiine and Thymidine Supplement and Penicillin-Streptomycin (at 50 μg streptomycin/ml, Gibco BRL). Membrane fragments were prepared using cell lysis/differential centrifuigation methods as described previously (Siciliano et al., 1990, J. Biol. Chem., 265, 19658). Protein concentration was estimated by BCA protein assay (Pierce, Rockford, Ill.) according to the manufacturer's instructions.

iii) Assay

¹²⁵I MCP-1 was prepared using Bolton and Hunter conjugation (Bolton et al., 1973, Biochem. J., 133, 529; Amersham International plc]. Equilibrium binding assays were carried out using the method of Ernst et al., 1994, J. Immnunol., 152, 3541. Briefly, varying amounts of ¹²⁵I-labeled MCP-1 were added to 7 μg of purified CHO-CCR2B cell membranes in 100 μl of Binding Buffer. After 1 hour incubation at room temperature the binding reaction mixtures were filtered and washed 5 times through a plate washer (Brandel MLR-96T Cell Harvester) using ice cold Binding Buffer. Filter mats (3randel GF/B) were pre-soaked for 60 minutes in 0.3% polyethylenimine prior to use. Following filtration individual filters were separated into 3.5 ml tubes (Sarstedt No. 55.484) and bound ¹²⁵I-labeled MCP-1 was determined (LKB 1277 Gammamaster). Cold competition studies were performed as above using 100 pM ¹²⁵I-labeled MCP-1 in the presence of varying concentrations of unlabelled MCP-1. Non-specific binding was determined by the inclusion of a 200-fold molar excess of unlabelled MCP-1 in the reaction.

Ligand binding studies with membrane fragments prepared from CHO-CCR2B cells showed that the CCR2B receptor was present at a concentration of 0.2 pmoles/mg of membrane protein and bound MCP-1 selectively and with high affinity (IC₅₀=110 pM, K_{d}=120 pM). Binding to these membranes was completely reversible and reached equilibrium after 45 minutes at room temperature, and there was a linear relationship between MCP-1 binding and CHO-CCR2B cell membrane concentration when using MCP-1 at concentrations between 100 pM and 500 pM.

Test compounds dissolved in DMSO (5 μl) were tested in competition with 100 pM labelled MCP-1 over a concentration range (0.01-50pM in duplicate using eight point dose-response curves and IC₅₀ concentrations were calculated.

Compounds tested of the present invention had IC₅₀ values of 50 μM or less in the hMCP-1 receptor binding assay described herein.

b) MCP-1 Mediated Calcium Flux in THP-1 Cells

The human monocytic cell line TIP-1 was grown in a synthetic cell culture medium RPM 1640 supplemented with 10% foetal calf serum, 6 mM glutamine and Penicillin-Streptomycin (at 50 μg streptomycin/ml, Gibeo BRL). THP-1 cells were washed in HBSS (lacking Ca²⁺ and Mg²⁺)+1 mg/ml BSA and resuspended in the same buffer at a density of 3×10⁶ cells/ml. The cells were then loaded with 1 mM FURA-2/AM for 30 mrin at 37° C., washed twice in HBSS, and resuspended at 1×10⁶ cells/ml. TrP-1 cell suspension (0.9 ml) was added to a 5 ml disposable cuvette containing a magnetic stirrer bar and 2.1 ml of prewarmed (37° C.) HBSS containing 1 mg/ml BSA, 1 mM MgCl₂ and 2 mM CaCl₂. The cuvette was placed in a fluorescence spectrophotometer (Perkin Elner, Norwalk, Conn.) and preincubated for 4 min at 37° C. with stirring. Fluorescence was recorded over 70 sec and cells were stimulated by addition of hMCP-1 to the cuvette after 10 sec. [Ca²⁺]i was measured by excitation at 340 nm and 380 nm alternately and subsequent measurement of the intensity of the fluorescence emission at 510 nm. The ratio of the intensities of the emitted fluorescent light following excitation at 340 nm and 380 nm, (R), was calculated and displayed to give and estimate of cytoplasmic [Ca²⁺] according to the equation:

$$[ {Ca}^{2 +} ] i = {K}_{d} \frac{( R - R min )}{( R max - R )} ( Sf2 / Sb2 )$$

where the K_{d} for FURA-2 Ca²⁺ complex at 37° C. was taken to be 224 nm. Rₘₐₓ is the maximal fluorescence ratio determined after addition of 10 mM lonomycin, Rₘᵢₙ is the minimal ratio determined by the subsequent addition of a Ca²⁺ free solution containing 5 mM EGTA, and Sf2/Sb2 is the ratio of fluorescence values at 380 nm excitation determined at Rₘᵢₙ and Rₘₐₓ, respectively.

Stimulation of THP-1 cells with hMCP-1 induced a rapid, transient rise in [Ca²⁺]i in a specific and dose dependent manner. Dose response curves indicated an approximate EC₅₀ of 2 nm. Test compounds dissolved in DMSO (10 μl) were assayed for inhibition of calcium release by adding them to the cell suspension 10 sec prior to ligand addition and measuring the reduction in the transient rise in [Ca²⁺]i. Test compounds were also checked for lack of agonist activity by addition in place of bMCP-1.

c) hMCP-1 and RANTES Mediated Chemotaxis.

In vitro chemotaxis assays were performed using the human monocytic cell line THP-1. Cell migration through polycarbonate membranes was measured by enumerating those passing through either directly by Coulter counting or indirectly by use of a colourimetric viability assay measuring the cleavage of a tetrazolium salt by the mitochondrial respiratory chain (Scudiero D. A. et al. 1988, Cancer Res., 48, 4827-4833).

Chemoattractants were introduced into a 96-well microtitre plate which forms the lower well of a chemotaxis chamber fitted with a PVP-free 5 μm poresize polycarbonate adhesive framed filter membrane (NeuroProbe MB series, Cabin John, Md. 20818, USA) according to the manufacturer's instructions. The chemoattractant was diluted as appropriate in synthetic cell culture medium, RPMI 1640 (Gibco) or supplemented with 2 mM glutamine and 0.5% BSA, or alternatively with HBSS with Ca²⁺ and Me²⁺ without Phenol Red (Gibeo) plus 0.1% BSA. Each dilution was degassed under vacuum for 30 min and was placed (400 μl) in the lower wells of the chamber and TB-1 cells (5×10⁵ in 100 μl RPMI 1640+0.5%BSA) were incubated in each well of the upper chamber. For the inhibition of chemotaxis the chemoattractant was kept at a constant submaximal concentration determined previously (1 nM MCP-1) and added to the lower well together with the test compounds dissolved in DMSO (final DMSO concentration <0.05% v/v) at varying concentrations. The chamber was incubated for 2 h at 37° C. under 5% CO₂. The medium was removed from the upper wells which were then washed out with 200 μl physiological saline before opening the chamber, wiping dry the membrane surface and centrifuging the 96-well plate at 600 g for 5 min to harvest the cells. Supernatant (150 μl) was aspirated and 10 μl of cell proliferation reagent, WST-1, {4-[3-(4-iodophenyl)-2-(4-nitrophenyl)-2H-5-tetrazolio]-1,3-phenyl disulfonate} plus an electron coupling reagent (Boehringer Mannheim, Cat.no. 1644 807) was added back to the wells. The plate was incubated at 37° C. for 3 h and the absorbance of the soluble formazan product was read on a microtitre plate reader at 450 nm. The data was input into a spreadsheet, corrected for any random migration in the absence of chemoattractant and the average absorbance values, standard error of the mean, and significance tests were calculated. bMCP-1 induced concentration dependent cell migration with a characteristic biphasic response, maximal 0.5-1.0 nm.

In an alternative form of the above assay, fluorescently tagged cells can be used in order to assist in end point detectioln. lthis case, the TP-1 cells used are fluorescently tagged by incubation in the presence of 5 mM Calcein AM (Glycine, N,N′-[[3′,6′-bis(acetyloxy)-3-oxospiro[isobenzofaran-1(3H),9′-[9H]xanthene]-2′,7′-diyl]bis(methylene)] bis[N-[2-[(acetyloxy)mnethoxy]-2-oxoethyl]]-bis[(acetyloxy)methyl] ester; Molecular Probes) for 45 minutes in the dark. Cells are harvested by centrifligation and resuspended in 1BSS (without Phenol Red) with Ca²⁺, Me²⁺ and 0.1% BSA. 50 μl (2×105 cells) of the cell suspension are placed on the filter above each well and, as above, the unit is incubated at 37° C. for 2 hours under 5% CO₂. At the end of the incubation, cells are washed off the upper face of the filter with phosphate buffered saline, the filter removed from the plate and the number of cells attracted to either the underside of the filter or the lower well estimated by reading fluorescence at 485 nm excitation, 538 nm emission wavelengths (finax, Molecular Devices). The data was input into a spreadsheet, corrected for any random migration in the absence of chemoattractant and the average fluorescence values, standard error of the mean, percentage inhibition and IC₅₀ of compounds under test and significance tests can be calculated. In addition to MCP-1 induced chemotaxis, this alternative form of the assay was also used to measure inhibition of RANTES (2 nM) induced chemotaxis.

d) Binding to Human Peripheral Blood Mononuclear Cells(PBMCs)

i) Preparation of Human PBMCs

Fresh human blood (200 ml) was obtained from volunteer donors, collected into sodium citrate anticoagulant to give a final concentration of 0.38%. The blood was mixed with Sedimentation Buffer and incubated at 37° C. for 20 minutes. The supernatant was collected and centrifuged at 1700 rpm for 5 minutes (Sorvall RT6000D). The pellet obtained was resuspended in 20 ml RPMI(BSA (lrg/ml) and 4×5 mls of cells were carefully layered over 4×5 mls of Lymphoprep™ (Nycomed) in 15 ml centrifuge tubes. Tubes were spun at 1700 rpm for 30 minutes (Sorvall RT6000D) and the resultant layer of cells was removed and transferred to 50 ml Falcon tubes. The cells were washed twice in Lysis Buffer to remove any remaining red blood cells followed by 2 washes in RPMI/BSA. Cells were resuspended in 5 mls of Binding Buffer. Cell number was measured on a Coulter counter and additional binding buffer was added to give a final concentration of 1.25×10⁷ PBMCs /ml.

ii) Assay

[¹²⁵I]MCP-1 was prepared using Bolton and Hunter conjugation (Bolton et al., 1973, Biochem. J., 133, 529; Amersham International plc]. Equilibrium binding assays were carried out using the method of Ernst et al., 1994, J. Immunol., 152, 3541. Briefly, 50 μl of ¹²⁵I-labeled MCP-1 (final concentration 100 pM) was added to 40 μl (5×10⁵ cells) of cell suspension in a 96 well plate. Compounds, diluted in Whole Cell Binding Buffer from a stock solution of 10 mM in DMSO were added in-a final volume of 5 μl to maintain a constant DMSO concentration in the assay of 5%. Total binding was determined in the absence of compound. Non-specific binding was defined by the addition of 51 μl cold MCP-1 to give a final assay concentration of 100 nM. Assay wells were made up to a final volume of 100 μl with Whole Cell Binding Buffer and the plates sealed. Following incubation at 37° C. for 60 minutes the binding reaction mixtures were filtered and washed for 10 seconds using ice cold Wash Buffer using a plate washer (Brandel MLR-96T Cell Harvester). Filter mats (Brandel GF/B) were pre-soaked for 60 minutes in 0.3% polyethylenimine plus 0.2% BSAprior to use. Following filtration individual filters were separated into 3.5 ml tubes (Sarstedt No. 55.484) and bound ¹²⁵I-labeled MCP-1 was determined (LKB 1277 Gammamaster).

Test compound potency was determined by assay in duplicate using six point dose-response curves and IC₅₀ concentrations were determined.

No physiologically unacceptable toxicity was observed at the effective dose for compounds tested of the present invention. The invention is further illustrated, but not limited by the following Examples in which the following general procedures were used unless stated otherwise.

i) N,N-Dimethylformamide (NMF) was dried over 4 Å molecular sieves. Anhydrous tetrahydroftran (THF) was obtained from Aldrich SURESEAL™ bottles. Other commercially available reagents and solvents were used without further purification unless otherwise stated. Organic solvent extracts were dried over anhydrous MgSO₄.

ii) ¹H, ¹³C and ¹⁹F NMR were recorded on Bruker WM200, WM250, WM300 or WM400 instruments using DMSO-d₆ with Me₄Si or CCl₃F as internal standard as appropriate, unless otherwise stated. Chemical shifts are quoted in δ (ppm) and peak multiplicities are designated as follows: s, singlet; d, doublet; dd, doublet of doublets; t, triplet; dt, doublet of triplets; q, quartet; m, multiplet; br, broad.

iii) Mass spectra were recorded on VG 12-12 quadrupole, VG 70-250 SE, VG ZAB 2-SE or a VG modified AEI/Kratos MS9 spectrometers.

iv) For TLC analysis, Merck precoated TLC plates (silica gel 60 F254, d=0.25 mm) were used.

v) Flash chromatography was performed on silica (Merck Kieselgel: Art.9385).

### EXAMPLE 1

N-(3-trifluoromethyl-4-chlorobenzyl -5-hydroxyindole-2-carboxylic acid

Sodium hydroxide (1M, 100 ml) was added to a stirred solution of ethyl N-(3-trifluoromethyl-4-chlorobenzyl)-5-acetoxyindole-2-carboxylate (11.82 g) in water (50 ml) and methanol (150 ml). The reaction was stirred at 55° C. for 6 hours. The methanol was removed under vacuo and the remaining solution was acidified by the addition of aqueous hydrochloric acid (2M, 50 ml) precipitating the product as a white solid. The product was filtered, washed with water and dried in vacuo to yield a cream solid (9.53 g,) which was purified by column chromatography using ethylacetate as the eluant. Crystallisation from methanol/water yielded the title compound as a cream solid (7.08 g,71%) NMR: (CD₃SOCD₃) δ 5.84 (s, 2H), 6.83 (dd, 1H), 6.95 (d, 1H), 7.11-7.19 (m, 2H), 7.36 (d, 1H), 7.55-7.64 (m, 2H), 9.03 (s, 1H); m/z 368 (M−H).

The procedure described in the above example was repeated using the appropiate indole ester as the starting materials. Thus were obtained the compounds described below.

### EXAMPLE 2

N-(3-fluoro-4-trifluoromethylbenzyl)-5-hydroxyindole-2-carboxylic acid

50% yield. NMR (CD₃SOCD₃) δ 5.87 (s, 2H), 6.85 (m, 2H), 6.99 (dd, 1H), 7.11 (d, 1H), 7.17 (s, 1H), 7.33 (d, 1H), 7.67 (t, 1H); m/z 352 (M−H⁺).

### EXAMPLE 3

N-(3-chloro-4-trifluoromethylbenzyl -5-hydroxyindole-2-carboxylic Acid

(55% yield). NMR(CD₃SOCD₃) δ: 5.9 (s, 2H), 6.9 (m, 1H), 7.1 (m, 2H), 7.25 (s, 1H), 7.4 (m, 2H), 7.8 (d, 1H), 9.1 (s, 1H); m/z 368/370 (M−H⁺).

### EXAMPLE 4

N-(3-bromo-4-chlorobenzyl)-5-hydroxyindole-2-carboxylic Acid

71% yield. NMR (CD₃SOCD₃) δ 5.76 (s, 2H), 6.80 (d, 1H), 6.95 (m, 2H), 7.12 (s, 1H), 7.36 (d, 1H), 7.40 (s, 1H), 7.47 (d, 1H), 9.00 (s, 1H); m/z 380 (MH⁺).

### EXAMPLE 5

N-(3-fluoro-4-bromobenzyl -5-hydroxyindole-2-carboxylic Acid

53% yield. NMR (CD₃SOCD₃) δ 5.77 (s, 1H), 6.70 (d, 1H), 6.80 (dd, 1H), 6.96 (s, 1H), 7.00 (d, 1H), 7.17 (s, 1H), 7.32 (d, 1H), 7.57 (t, 1H), 9.00 (s, 1H), 12.82 (s, 1H); m/z 362 (M−H⁺).

### EXAMPLE 6

N-(3-bromo-4-fluorobenzyl)-5-hydroxyindole-2-carboxylic Acid

55% yield. NMR (CD₃SOCD₃) δ 5.77 (s, 2H), 6.80 (dd, 1H), 6.97 (d, 1H), 6.99 (m, 1H), 7.13 (s, 1H), 7.23 (t, 1H), 7.38 (m, 2H), 9.00 (s, 1H); m/z 362 (M−H⁺).

### EXAMPLE 7

N-(3-trifluoromethyl-4-fluorobenzyl)-4-fluoro-5 hydroxyindole-2-carboxylic Acid

(58% yield). NMR(CD₃SOCD₃) δ: 5.85 (s, 2H), 7.0 (t, 1H), 7.1 (m,2H), 7.2-7.3 (m,3H), 7.4 (t, 1H), 7.95 (dd, 1H), 9.3 (s,1H) 13.1 (s, 1H); m/z 370 (M−H⁺).

### EXAMPLE 8

N-(3-trifluoromethyl-4-chlorobenzyl)-4-fluoro-5-hydroxyindole-2-carboxylic Acid

97% yield. NMR (CD₃SOCD₃) δ 5.80 (s, 2H), 7.00 (t, 1H), 7.16 (dd, 1H), 7.20 (m, 2H), 7.60 (m, 2H), 9.30 (s, 1H); m/z 386 (MH⁺).

### EXAMPLE 9

N-(3-trifluoromethyl-4-fluorobenzyl)-4,6-difluoro-5-hydroxyindole-2-carboxylic Acid

83% yield. NMR (CD₃SOCD₃) δ 5.80(s, 2H), 7.20(s, 1H), 7.23(m, 1H), 7.30-7.50(m, 2H), 7.58(m, 1H), 9.60(s, 1H); M/z(−) 388.2 (M−H⁺)

### EXAMPLE 10

N-(3,4-chlorobenzyl)-4,6-dichloro-5-hydroxyindole-2-carboxylic Acid

82% yield. NMR (CD₃SOCD₃) δ 5.92 (s, 2H), 6.87 (s, 1H), 6.99 (dd, 1H), 7.37 (d, 1H), 7.5 (d, 1H), 7.55 (s, 1H); m/z 406,404,402 (M−H⁺)

### EXAMPLE 11

N-(3-trifluoromethyl-4-fluorobenzyl -3-bromo-5-hydroxyindole-2-carboxylic Acid

(92% yield). NMR(CD₃SOCD₃) δ: 5.8 (s, 2H), 6.9 (m, 2H), 7.25 (dd,1H), 7.35-7.55 (m,2H), 7.6 (dd, 1H), 9.4 (s,1H); m/z 430/432 (M−H⁺).

### EXAMPLE 12

N-(3-trifluoromethyl-4-chlorobenzyl -3-bromo-5-hydroxyindole-2-carboxylic Acid

(309 mg, 87%). NMR(CD₃SOCD₃) δ:: 5.85 (s, 21, 6.8-7.0 (m, 3H), 7.4 (d, 1H), 7.75 (d, 1H), 9.4 (s,1H); m/z 446/448 (M−H⁺).

### EXAMPLE 13

N-(3-chloro-4-trifluoromethylbenzyl)-3-bromo-5-hydroxyindole-2-carboxylic Acid

(82% yield) NMR(CD₃SOCD₃) δ:: 5.8 (s, 2H), 6.9 (m, 2H), 7.1 (dd,1H), 7.45 (d, 1H), 7.6 (m, 2H), 9.4 (s,1H); m/z 447 (M−H⁺).

### EXAMPLE 14

N-(3-fluoro-4-trifluoromethylbenzyl)-3-chloro-5-hydroxyindole-2-carboxylic Acid

NMR (CD₃SOCD₃) δ 5.8 (s,2H), 6.9(m,2H), 7.25(m,1H),7.4(m,2H), 7.6(d,1H), 9.4(s,1H); m/z 386.0(M−H⁺).

### EXAMPLE 15

N-(3-fluoro-4-trifluoromethylbenzyl)-3-iodo-5-hydroxyindole-2-carboxylic Acid

NMR (CD₃SOCD₃) δ 5.8 (s,2H), 6.8(s,1H), 6.9(d,1H), 7.2(m,1H), 7.4(m,2M, 7.6(d,1H), 9.3(s, 1H); m/z 478(M−H⁺).

### EXAMPLE 16

N-(3-trifluoromethyl-4chlorobenzyl)-3-methoxy-5-hydroxyindole-2-carboxylic Acid

(108% yield as hydrate) NMR: 3.9 (s,3H) 5.7 (s, 2H), 6.8 (dd, 1H), 6.9 (d,1H), 7.2(d, 1H) 7.4 (d, 1H), 7.6 (m, 2H), 9.1 (s,1H); m/z 398 (M−H⁺).

### EXAMPLE 17

N-(3-trifluoromethyl-4-fluorobenzyl)-5-hydroxy-6chloroindole-2-carboxylic Acid

(68% yield). NMR (CD₃SOCD₃) δ 5.8 (s, 2H), 7.1-7.2 (m, 3H), 7.4-7.55 (m, 2H), 7.7 (s, 1H), 9.8 (s, 1H); m/z 386 (M−H⁺).

### EXAMPLE 18

N-(3-trifluoromethyl-4-chlorobenzyl)-5-hydroxy-6-chloroindole-2-carboxylic Acid

71% yield. NMR (CD₃SOCD₃) δ 5.74 (s, 2H), 7.04-7.21 (m, 3H), 7.53-7.63 (m, 2H), 7.7 (s, 1H), 9.72 (bs, 1H); m/z 402.1/404.5 (M−H⁻)

### EXAMPLE 19

N-(3-trifluoromethyl-4-chlorobenzyl)-5-hydroxy-7-fluoroindole-2-carboxylic Acid

55% yield. NMR (CD₃SOCD₃) δ 5.90 (s, 2H), 6.60 (m, 1H), 6.80 (m, 1H), 7.15 (m, 2H), 7.52(m, 1H), 7.60 (d, 1H); M/z(−) 385.85 (M−H⁺)

### EXAMPLE 20

N-(3-trifluoromethyl-4-chlorobenzyl -5-hydroxy-6-bromoindole-2-carboxylic Acid

90% yield. NMR (CD₃SOCD₃) δ 5.82 (s, 2H), 7.10(m, 1H), 7.18 (d, 2H), 7.60 (m, 2H), 7.82(s, 1H), 9.80 (s, 1H), 13.0 (s, 1H); m/z 446.18 (M−H⁺)

### EXAMPLE 21

N-(3,4-dichlorobenzyl)-5-hydroxy-6-bromoindole-2-carboxylic Acid

92% yield. NMR (CD₃SOCD₃) δ 5.80 (s, 2H), 6.85 (m, 1H), 7.15 (s, 2H), 7.25 (m, 1H), 7.50 (d, 1H), 7.80 (s, 1H), 9.80 (s, 1H); m/z 412.1 (M−H⁺)

### EXAMPLE 22

N-(3-trifluoromethyl-4-chlorobenzyl -5-hydroxy-6-fluoroindole-2-carboxylic Acid

97% yield. NMR (CD₃SOCD₃) δ 5.8 (s, 2H), 7.1-7.2 (m, 3H), 7.49 (d, 1H), 7.55-7.63 (m, 2H), 9.49 (s, 1H), 12.86 (bs, 1H); m/z 386, 388 (M−H⁺)

### EXAMPLE 23

N-(3,4-dichlorobenzyl)-5-hydroxy-6-fluoroindole-2-carboxylic Acid

97% yield NMR (CD₃SOCD₃) δ 5.75 (s, 2H), 6.9 (dd, 1H), 7.1-7.2 (m, 2H), 7.3 (d, 1H), 7.45 (d, 1H), 7.5 (d, 1H), 9.5 (bs, 1H); m/z 353 (M−H⁺)

### EXAMPLE 24

N-(3,4-dichlorobenzyl)-5-hydroxy-6-chloroindole-2-carboxylic Acid

41% yield. NMR (CD₃SOCD₃) δ 5.8 (s, 2H), 6.9 (dd, 1H), 7.2 (s, 2H), 7.3 (d, 1H), 7.5 (d, 1H), 7.65 (s, 1H), 9.75 (s, 1H); m/z 398, 396 M-H⁺)

### EXAMPLE 25

N-(3-trifluoromethyl-4-chlorobenzyl)-4-chloro-5-hydroxyindole-2-carboxylic Acid

93% yield. NMR (CD₃SOCD₃) δ 5.86 (s, 2H), 7.01 (d, 1H), 7.09-7.13 (m, 2H), 7.4 (d, 1H), 7.58-7.68 (m, 2H), 9.66 (bs, 1H); m/z 402, 404 (M−H⁺).

### EXAMPLE 26

N-(3-trifluoromethyl-4-chlorobenzyl)-4,6-dichloro-5-hydroxyindole-2-carboxylic Acid

76% yield. NMR (CD)₃SOCD₃) δ 5.86 (s, 2H), 7.09 (dd, 1H), 7.15 (s, 1H), 7.59 (d, 1H), 7.64 (d, 1H), 7.81 (s, 1H), 9.64 (bs, 1H); m/z 392, 394 (M−H⁺).

### EXAMPLE 27

N-(3-trifluoromethyl-4-chlorobenzyl)-5-acetoxyindole-2-carboxylic Acid Prodrug of compound No. 1 of Example 1)

To a solution of N-(3-trifluoromethyl-4-chlorobenzyl)-5-hydroxyindole-2-carboxylic acid (1.01 g) in warm ethyl acetate (80 ml) was added 4-dimethylaminopyridine (30 mg) and acetic anhydride (0.64 ml) and the resulting mixture was stirred for 18 hours. The organics were washed with 1N HCl and dried. The organics were concentrated and purified by column chromatography, eluting with ethyl acetate to give the desired product(808 mg, 72%). ¹H NMR (MSO-d₆) δ 2.25 (s, 3H), 5.9 (s, 2H), 7.05 (m, 1H), 7.15 (m, 1H), 7.32 (s, 1H), 7.43 (d, 1H), 7.60 (m, 2H), 7.65 (d, 1H); m/z 410 M−H⁺).

Preparation of Starting Materials

The starting materials for the Examples above are either commercially available or are readily prepared by standard methods from known materials. For example the following reactions Methods A-E) are illustrations but not limitations of the preparation of the starting materials used in the above reactions.

Method A

Ethyl 5-acetoxy-N-(3-trifluoromethyl-4-chlorobenzylindole-2-carboxylate

i) Ethyl 5-hydroxyindole-2-carboxylate

Boron tribromide (64.58 g) was added dropwise to a stirred solution of ethyl 5-methoxyindole-2-carboxylate (20 g) in dichloromethane (1000 ml) at −78° C. under an atmosphere of argon. The reaction was allowed to warm to room temperature and stirred for a further 2 hours. The reaction was poured into ice/saturated aqueous sodium hydrogen carbonate solution with stirring and extracted with ethyl acetate. Combined organic extracts were washed with saturated aqueous sodium hydrogen carbonate solution, water, aqueous saturated sodium chloride solution and dried. The solution was concentrated in vacuo and the residue was purified by column chromatography using 0-60% diethyl ether: iso-hexane as eluent to yield product as a white solid (9.02 g, 48%). NMR(CD₃SOCD₃): δ 1.31 (t, 3H), 4.29 (q, 2H), 6.79 (dd, 1H), 6.90 (dd, 1H), 7.22 (d, 1H), 8.84 (s, 1H), 11.52 (brs, 1H); m/z 206 (MH⁺).

ii) Ethyl 5-acetoxyindole-2-carboxylate

A stirred solution of ethyl 5-hydroxyindole-2-carboxylate (7.79 g) and 4-dimethylaminopyridine (20 mg) in acetic anhydride (80 ml) was heated at 80° C. for 4 hours. The reaction was concentrated in vacuo and the residue was dissolved in ethyl acetate. Combined organic extracts were washed with hydrochloric acid (2 M), saturated aqueous sodium hydrogen carbonate solution, water, aqueous saturated sodium chloride solution and dried. The solution was concentrated in vacuo to yield the product as a yellow solid (9.39 g,100%). NMR(CD₃SOCD₃): δ 1.20 (t, 3H), 2.10 (s, 3H), 4.19 (q, 2H), 6.86 (dd, 1H), 6.97 (d, 1H), 7.20 (s, 1H), 7.29 (d, 1H); m/z 248 (MH⁺).

iii) Ethyl 5-acetoxy-N-(3-trifluoromethyl-4-chlorobenzyl)indole-2-carboxylate

Sodium hydride (1.78 g) was added to a stirred solution of ethyl 5-acetoxyindole-2-carboxylate (10 g) and 3-trifluoromethyl-4-chlorobenzylbromide (11.64 g) in DMF (200 ml) under an atmosphere of argon. The reaction was stirred at ambient temperature for 16 hours, then concentrated in vacuo and the residue partitioned between ethyl acetate and water. Combined organic extracts were dried, concentrated under vacuo and purified by column chromatography using i-hexane-15%ethylacetate/isohexane as the eluant to yield a cream solid. Crystalisation from ethylacetate/isohexane yielded the product as a cream solid.(13.26 g, 74%/). NMk (CD₃SOCD₃): δ 1.37 (t, 3H), 2.31 (s, 3H), 4.32 (q, 2H), 5.82 (s, 2H), 7.0-7.09 (m, 2H), 7.22-7.29 (m, 1H), 7.31-7.4 (m, 2H) 7.43 (d, 1H), 7.51 (s, 1H).

The procedures described in Method A i)-iii) were repeated using the appropriate benzyl halide. Thus were obtained the compounds described below.

Ethyl N-(3-fluoro-4-trifluoromethylbenzyl)-5-acetoxyindole-2-carboxylate

860 mg, 96% NMR (CDCl₃) δ 1.39 (t, 3H), 2.36 (s, 3H), 4.37 (q, 2H), 5.83 (s, 2H), 6.83 (d, 1H), 6.90 (d, 1H), 7.08 dd, 1H), 7.23 (s, 1H), 7.40 (s, 1H), 7.42 (d, 1H), 7.50 (t, 1H); m/z 424 (1H).

Ethyl N-(3-chloro-4-trifluoromethylbenzyl)-5-acetoxyindole-2-carboxylate

55% yield. NMR (CDCl₃) δ 1.4 (t,3H) 2.3 (s,3H) 4.3 (q, 2H) 5.8 (s, 2H), 6.95 (d, 1H), 7.1 (dd, 2H), 7.2 (m, 2H), 7.4 (s, 1H), 7.45 (d, 1H) 7.55 (d, 1H); m/z 440/422 (MH⁺).

Ethyl N-(3-bromo-4-chlorobenzyl -5-acetoxyindole-2-carboxylate

15% yield. NMR (CDCl₃) δ 1.37 (t, 3H), 2.30 (s, 3H), 4.31 (q, 2H), 5.74 (s, 2H), 6.83 (d, 1H), 7.03 (dd, 1H), 7.24 (m, 2H), 7.37 (m, 2H), 7.40 (d, 1M. m/z 449 (MH⁺).

Ethyl N-(3-fluoro-4-bromobenzyl)-5-acetoxyindole-2-carboxylate

77% yield. NMR (CDCl₃) δ 1.37 (t, 31, 2.30 (s, 3H), 4.37 (q, 2H), 5.77 (s, 2H), 6.72 (d, 1H), 6.74 (d, 1H), 7.03 (dd, 1H), 7.23 (m, 1H), 7.37 (s, 1H), 7.40 (t, 1H).

Ethyl N-(3-bromo-4fluorobenzyl -5-acetoxyindole-2-carboxylate

41% yield. NMR (CDCl₃) δ 1.40 (t, 3H), 2.34 (s, 3H), 4.37 (q, 2H), 5.72 (s, 2H), 6.95 (dd, 1H), 7.05 (dd, 1H), 7.23 (m, 2H), 7.37 (s, 1H), 7.40 (d, 1H), 7.62 (d, 1H); m/z 433 (MH+).

Method B

Methyl-N-(3-trifluoromethyl-fluorobenzyl)-4-fluoro-5-hydroxyindole-2-carboxylate

(i) 2-Fluoro-3-benzyloxybenzaldehyde

2-Fluoro-3-hydroxybenzaldehyde (16.49 g) was dissolved in dimethylformamide (200 ml) and stirred under an argon atmosphere. Sodium hydride was added (60% in mineral oil, 5.18 g) and the mixture was stirred for 30 minutes. Benzyl bromide was added (16.8 ml) and the mixture was stirred overnight. Reaction mixture was concentrated in vacuo and the resulting residue was partitioned between diethyl ether (200 ml) and water (200 ml). Combined organic extracts were washed with water (400 ml), dried (MgSO4) and concentrated in vacuo. The residue was purified by flash column chromatography, using a gradient of 0-10% ethyl acetate/iso-hexane as eluent to give the desired product as a yellow solid (18.41 g, 68%): ¹H NMR (CD₃SOCD₃) δ 5.20 (s, 2H), 7.2-7.6 (m, 8H), 10.21(s, 1H)

(ii) Methyl-2-azido-3-(2-fluoro-3-benzyloxyphenyl)propenoate

A mixture of methylazidoacetate (36.64 g) and 2-Fluoro-3-benzyloxy benzaldehyde (18.32 g) in methanol (250 ml) was added dropwise, with stirring, over 1 hour to a mixture of sodium methoxide (17.20 g) in methanol (100 ml) at −25° C. under a stream of argon. Mixture was left to stir for 20 minutes, allowed to warm to 5° C. and stirred overnight. The resulting precipitate was filtered, then washed sequentially with cold methanol, dilute solution of acetic acid in water and water. The resulting solid was dried under vacuum to give the product as a pale brown solid (16.70 g) which was used without purification.

(iii) Methyl-4-fluoro-5-benzyloxyindole-2-carboxylate

A solution of methyl-2-azido-3-(2-fluoro-3-benzyloxyphenyl)propenoate (16.7 g) in xylene (600 ml) was added dropwise with stirring to refluxing xylene (2.4L) over 1 hour and then stirred for a further 20 minutes. The reaction mixture was concentrated in vacuo and purified by flash column chromatography, using a gradient of 0-100% ethyl acetate/iso-hexane as eluent to give the product as a yellow solid (12.93 g, 54%). ¹H NMR (CD₃SOCD₃) δ 3.85(s, 3H), 5.15 (s, 2H), 7.05-7.45 (m, 8H), 12.06 (s, 1H); m/z 300.4 (MH⁺)

In a similar manner, steps (ii) and (iii) were repeated, but using 2-chloro-3-methoxybenzaldehyde and ethyl azidoacetate was prepared:—

Ethyl-4-chloro-5-methoxyindole-2-carboxylate

¹H NMR (CD₃SOCD₃) δ 1.31 (t, 3H), 3.84 (s, 3H), 4.32 (q, 2H),7.0 (d, 1H), 7.22 (d, 1H), 7.39 (d, M), 12.2 (bs, 1H).

(iv) Methyl-N-(3-trifluoromethyl-4-fluorobenzyl-4-fluoro-5-benzyloxyindole-2-carboxylate

Sodium hydride (60% in mineral oil, 75 mg) was added to a solution of methyl-4-fluoro-5-benzyloxyindole-2-carboxylate (257 mg) in dimethylformamide (10 ml) cooled to 5° C. and the mixture was stirred under an argon atmosphere for 30 minutes. 3-Trifluoromethyl-4-fluorobenzyl chloride (280 mg) was added and the mixture was allowed to warm to room temperature and then stirred for 4 hours. The reaction mixture was partitioned between ethyl acetate and water. The organic extracts were washed with water, dried (MgSO4), concentrated in vacuo and purified by flash column chromatography, using iso-hexane followed by 5% ethyl acetate/iso-hexane as eluent, to give the desired product (140 mg, 34/). ¹H NMR (CDCl₃) δ 3.9 (s, 3H), 5.15 (s, 2H), 5.75 (s, 2H), 6.9-7.2 (m, 4H), 7.3-7.5 (m, 71); m/z 476 (M+H⁺)

In a similar manner but using the appropiate indole and benzyl halide was prepared:—

Ethyl-N-(3-trifluoromethyl-4-chlorobenzyl -4chloro-5-methoxyindole-2-carboxylate

82% yield. NMR (CDCl₃) δ 1.4 (t, 3H), 3.95 (s, 3H), 4.35 9q, 2H), 5.8 (s, 2H), 7.0-7.2 (m, 3H), 7.3-7.5 (m, 3H).

(v) Methyl-N-(3-trifluoromethyl-4fluorobenzyl)-4-fluoro-5-hydroxyindole-2-carboxylate

A mixture of methyl-N-(3-trifluoromethyl-4-fluorobenzyl)-4-fluoro-5-benzyloxyindole-2-carboxylate (140 mg) and 5% Pd/C (50 mg) in ethyl acetate (10 ml) was stirred under a hydrogen atmosphere for 5 hours, filtered through celite, concentrated in vacuo and purified by flash column chromatography using a gradient of 10-25% ethyl acetate/iso-hexane as eluent to give the desired product (60 mg, 53%/). ¹H NMR (CDCl₃) δ 3.9 (s, 31i), 4.9 (d, 1H), 5.8 (s, 2H), 6.9-7.2 (m, 4H), 7.4 (m, 2H); m/z 384 (M−H⁺)

In a similar manner but using the appropiate benzyl halide was prepared:

Methyl-N-(3-trifluoromethyl-4-chlorobenzyl)-4-fluoro-5-hydrox indole-2-carboxylate

89% yield. M (CD₃SOCD₃) δ 3.80 (s, 3H), 5.92 (s, 2H), 7.05 (t, 1H), 7.11 (dd, 1H), 7.22 (m, 2H), 7.60 (m, 2H), 9.37 (s, 1H); m/z 401 (MH⁺).

In a similar manner but starting from 2,4-difluoro-3-hydroxybenzaldehyde was prepared

Ethyl N-(3-trifluoromethyl-4-fluorobenzyl)-4,6-difluoro-5-hydroxyindole-2-carboxylate

¹H NMR (CD₃SOCD₃) δ 3.80 (s, 3H), 5.80 (s, 2H), 7.20-7.60 (m, 5H), 9.70 (s, 1H); m/z 402.2 M−H⁺)

Ethyl-N-(3-trifluoromethyl-4-chlorobenzyl)-4-chloro-5-hydroxyindole-2-carboxylate was prepared from ethyl-N-(3-trifluoromethyl-4-chlorobenzyl)-4chloro-5-methoxyindole-2-carboxylate by using the method as described in E(iv). 42% yield.H NMR (CD₃SOCD₃) δ 1.39 (t, 3H), 4.32 (q, 2H), 5.37 (s, 1H), 5.79 (s, 2H), 6.99-7.11 (m, 3H), 7.31-7.39 (m, 2H), 7.47 (d, 1H); m/z 430,432 (M−H⁺)

Method C

Ethyl 5-acetoxy-3-bromoindole-2-carboxylate

N-Bromosuccinimide (0.14 g) was added to a stirred solution of ethyl 5-acetoxyindole-2-carboxylate (0.2 g) in DMF (3.0 ml). The reaction was stirred for 4 hours, then poured into water. The resulting precipitate was filtered and dried in vacuo to give the title compound as a white powder (0.23 g, 87%). NMR 1.38 (t, 3H), 2.23 (s, 3H), 4.38 (q, 2H), 7.10 (dd, 1H), 7.23 (d, 1H), 7.50 (d, 1H), 12.28 (bs, 1H); m/z 326 (M⁺).

Method C2

Ethyl 5-acetoxy-3-chloroindole-2-carboxylate

A solution of ethyl 5-acetoxyindole-2-carboxylate (500 mg) in dichloromethane (10 ml) was stirred at room temperature in the presence of N-chlorosuccinimide (297 mg) and potassium carbonate (279 mg) overnight. The resulting precipitate was collected by filtration, washed with cold dichloromethane followed by water and dried under vacuum overnight to give the desired product as a white powder (425 mg, 75%). NMR: 1.35 (t,3H), 2.25 (s,3H), 4.4 (q,2H), 7.1 (d,1H), 7.3 (s,1H), 7.5 (d,1H), 12.2 (s,1H); m/z 281.9. (MH⁺).

Method C 3

Ethyl 5-acetoxy-3-iodoindole-2-carboxylate

A solution of ethyl 5-acetoxyindole-2-carboxylate (1 g) in dimnethylformamide (2 ml) was stirred at room temperature in the presence of potassium carbonate (1.12 g) and iodine (1.029 g) for 18 hours. The reaction was diluted with water (30 ml) and the resulting solid was filtered, washed with water and dried to give the desired product 1.32 g, 87%). NMR: 6 (CD₃SOCD₃) 1.4 (t, 3H), 4.4 (q,2H), 7.1 (d,1H), 7.15 (s,1H), 7.45 (d1H), 12.3 (s,1H): m/z 372(M−H⁻)

Ethyl N-(3-fluoro-4-trtifluoromethylbenzyl -5-acetoxy-3-iodoindole-2-carboxylate

To a solution of ethyl 5-acetoxy-3-iodoindole-2-carboxylate (400 mg) in dimethylformamide (15 ml) was added potassium carbonate (340 mg), tetrabutyl ammonium iodide (10 mg) and 4-fluoro-3-trifluoromethylbenzyl bromide (330 mg). The mixture was stirred for 18 hours. The mixture was diluted with water (10 ml) and extracted with ethyl acetate. The organic extracts were dried, concentrated and purified by column chromatography using 5% ethyl acetate/iso-hexane as the eluant to give the desired product (520 mg, 89%). NMR (CD₃SOCD₃): δ 1.3 (t, 3H), 2.25 (s, 3H), 4.3 (q, 2H), 5.85 (s, 2H), 7.2 (m, 3H), 7.4 (m, 1H), 7.8 (m, 2H): m/z 550 (MH⁺)

In a similar manner but using the appropiate ethyl 5-acetoxy-3-haloindole-2-carboxylate and benzyl halide were prepared:—

Ethyl N-(3-fluoro 4-trifluoromethylbenzyl)-5-acetoxy-3-chloroindole-2-carboxylate

70% yield. 458.1)

Ethyl N-(3-trifluoromethyl-4-fluorobenzyl)-5-acetoxy-3-bromoindole-2-carboxylate

96% yield. NMR (CDCl₃) δ 1.4 (t, 3H), 2.3 (s, 3H), 4.4 (q, 2H), 5.75 (s, 2H), 7.0-7.2 (m, 3H), 7.3 (m, 1H), 7.4 (m. 2H); m/z 502/504 (MH⁺).

Ethyl N-(3-trifluoromethyl-4-chlorobenzyl)-5-acetoxy-3-bromoindole-2-carboxylate

79% yield. NMR (CDCl₃) δ 1.4 (t, 3H), 2.35 (s, 3H), 4.4 (q, 2H), 5.8 (s, 2H), 7.05 (d, 1H), 7.1 (dd, 1H), 7.3 (m, 1H), 7.4 (d, 1H), 7.5 (m, 1H), 7.6 (s, 1H); m/z 518/520 (MH⁺)

Ethyl N-(3-chloro-4-trifluoromethylbenzyl)-5-acetoxy-3-bromoindole-2-carboxylate

63% yield. NMR (CDCl₃) δ 1.4 (t, 3H), 2.35 (s, 3H), 4.4 (q, 2H), 5.8 (s, 2H), 6.95 (d, 1H), 7.1 (dd, 1H), 7.25 (m, 2H), 7.5 (m, 1H), 7.6 (d, 1H); m/z 518/520 (MH⁺).

Method D

Ethyl 1-(3-trifluoromethyl-1-4-chlorobenzyl-3-methoxy-5hydroxyindole-2-carboxylate

(i) Ethyl 5-acetoxyindole-2-carboxylate

A mixture of ethyl 3-benzyloxyindole-2-carboxylate (10 g), cyclohexene (50 ml) and 10% palladium on carbon (2 g) in ethyl acetate (500 ml) was refluxed for 4 hours. The mixture was cooled and filtered through Celite. Acetic anhydride (5 ml) and N-dimethylaminopyridine (0.1 g) was added and the mnixture was refluxed for 15 mins. The mixture was cooled and ethanol was added to destroy excess acetic anhydride. The mixture was concentrated and the residue was recrystallised from ethyl acetate/iso-hexane to give the desired product as white needles (6.44 g, 77%) NMR (CD₃SOCD₃) δ 1.33 (t, 3H), 2.23 (s, 3H), 4.32 (q, 2H), 7.0 (dd, 1H), 7.13 (s, 1H), 7.38 (d, 1H), 7.42 (d, 1H), 11.93 (bs, 1H); m/z (M−H⁺)

(ii) Ethyl 5-acetoxy diazoindole-2-carboxylate

To a solution of ethyl 5-acetoxyindole-2-carboxylate (5 g) was added sodium nitrite (20 g) followed dropwise by glacial acetic acid (20 ml). After about half addition, brown fumes were evolved. The mixture was cooled to −10° C. and the remainder of the acetic acid was added. The mixture was allowed to stir for 18 hours. A further amount of sodium nitrite (10 g) and acetic acid (10 ml) was added and the resulting mixture was stirred for 18 hours. The mixture was partitioned between ethyl acetate and water. The organic extracts were separated, dried and concentrated to low volume. Hexane was added and the resulting solid was filtered to give the desired product (5.2 g, 94%). NMR (CDCl₃) δ 0.8 (t, 3H), 4.5 (q, 2H), 7.1 (dd, 1H), 7.4 (d, 1H), 8.0 (d, 1H); m/z 273 (M+H⁺)

(iii) Ethyl 3-methoxy-5-acetoxyindole-2-carboxylate

To a solution of ethyl 5-acetoxy diazoindole-2-carboxylate (4.6 g) in 1,2-dichloroethane was added methanol (10 ml), followed by a catalytic amount of rhodium (II) acetate dirner and the resulting mixture was refiuxed for 18 hours. The mixture was concentrated and the resulting residue was purified by column chromatography using 20% ethyl acetate/isohexane as eluent to give the desired product, which was purified firrther by titration with diethyl ether (2.34 g, 50%). NMR (CDCl₃) δ 1.4 (t, 3H), 2.3 (s, 3H), 4.05 (s, 3H), 4.4 (q, 2H), 7.05 (dd, 1H), 7.2-7.25 (m, 2H), 7.45 (d, 1H), 8.4 (bs, 1H); m/z 278.4 (M+H⁺).

Method E

Ethel N-(3-trifluoromethyl-4-chlorobenzyl -5-hydroxy-6-chloroindole-2 carboxylate

(i) Ethyl 2-acetyl-2-N′-(3-chloro-methoxylphenyl)hydrazino)propionate

Ethereal HCl (60 ml) was added to a solution of 3-chloro-p-anisidine in ethyl acetate (300 ml) to precipitate the salt, which was isolated by filtration and air dried. The salt (18.5 g) was suspended in 1.5 N HCl (230 ml) at −5° C. under argon. A solution of sodium nitrite (6.9 g) in water (50 ml) was added over 15 minutes to form a solution/slurry, which was stirred at −5° C. for a further 1 hour. (solution A)

A solution of sodium hydroxide (5.36 g) in water (10 ml) was added to a solution of ethyl-2-methylacetoacetate (13.5 ml) in ethanol (80 ml) at 5° C. The reaction was stirred at 5° C. for a further 1 hour and the pH was then adjusted to 4 by addition of sodium acetate (20 g).(solution B)

Solution B was added to solution A at −5° C. and the mixture was allowed to warm to ambient temperature over 3 hours before partitioning between water (250 ml) and ethylacetate (250 ml). The organic phase was dried (MgSO4), concentrated under vacuo and purified by column chromatography using 15% ethylacetate/isohexane as the eluant to yield the desired product (7 g, 21%); NMR (CDCl₃) δ 1.24 (t, 3H), 1.63 (s, 3H), 2.34 (s, 3), 3.98 (s, 3H), 4.22-4.35 (m, 2H), 7.02 (d, 1H), 7.72 (dd, 1H), 7.83 (d, 1H) m/z 270(M-CH3COH)⁺

In a similar manner but starting from 3-fluoro-4-methoxyaniline was prepared:

Ethyl 2-acetyl-2-(N′-(3-fluoro-4-methoxyphenyl)hydrazino) propionate

NMR (CD₃SOCD₃) S 1.25 (t, 3H), 1.55 (s, 3H), 2.35 (s, 3H), 4.0 (s, 3H), 4.2 (q, 2H), 7.4 (t, 1H), 7.5 (dd, 1H), 7.6 (d, 1H); m/z 255(MH⁺)

In a similar manner but starting from 3,5-dichloro-4-methoxyaniline was prepared:

Ethyl 2-(N′-(3,5-dichloro-4-methoxyphenylhydrazino propionate

NMR (CDCl₃) δ 1.4 (t, 3H), 2.05 (s, 3H), 3.85 (s, 3H), 4.3 (q, 2H), 7.13 (s, 2H), 7.52 9bs, 1H); m/z 307 MH⁺)

(ii) Ethyl 5-methoxy-6-chloroindole-2-carboxylate

A solution of ethyl 2-acetyl-2-{N′-(3-chloro-4-methoxyphenyl)hydrazino} propionate (1 g) and p-toluenesulphonic acid (1 g) in toluene (30 ml) was stirred at 100° C. for 18 hours. The mixture was then concentrated and purified by column chromatography using 15% ethylacetate/isohexane as the eluant to yield the desired product (70 mg, 8%); NMR (CDCl₃) δ 1.42 (t, 3H), 3.95, (s, 3H), 4.42 (q, 2H), 7.11 (s, 2H), 7.46 (s, 1H), 8.86 (bs, 1H)

In a similar manner but starting from ethyl 2-acetyl-2-(N′-(3-fluoro4-methoxyphenyl) hydrazino) propionate was prepared:

Ethyl 5-methoxy-6-fluoroindole-2-carboxylate N (CD₃SOCD₃) δ 1.3 (t, 3H), 3.8 (s, 3H), 4.3 (q, 2H), 7.1 (s, 1H), 7.2 (d, 1H), 7.3 (d, 1H); m/z 237 (MH⁺)

In a similar manner by starting from ethyl 2-(N′-(3,5-dichloro-4-methoxyphenyl) hydrazino) propionate was prepared:

Ethyl 5-methoxy-4,6-dichloroindole-2-carboxylate NMR (CD₃SOCD₃) δ 1.38 (t, 3H), 2.08 (s, 3H), 3.84 (s, 3H), 4.31 (q, 2), 7.23 (s, 2H), 7.5 (bs, 1H); m/z 307 (MH⁺)

(iii) Ethyl N-(3-trifluoromethyl-4-chlorobenzyl)-5-methoxy-6-chloroindole-2-carboxylate

Ethyl 5-methoxy-6-chloroindole-2-carboxylate was alkylated with 3-trifluromethyl-4-chlorobenzyl bromide using the methodology described in Method A(iii) to give the desired product (650 mg, 64%); NMR (CDCl₃) δ 1.36 (t, 3H), 3.93 (q, 2H), 5.75 (s, 2H), 7.01 (dd, 1H), 7.13 (s, 1H), 7.29 (s, 1H), 7.31 (s, 1H), 7.35 (d, 1H), 7.43 (d, 1H)

In a similar manner using the appropriate indole and benzyl halide were prepared:

Ethyl N-(3-trifluoromethyl-4-fluorobenzyl -5-methoxy-6-chloroindole-2-carboxylate

NMR (CD₃SOCD₃) δ 1.25 (t, 3H), 3.9 (s, 3H), 4.3 (q, 2H), 5.85 (s, 2H), 7.1-7.4 (m, 4H), 7.55 (d, 1H), 7.9 (s, 1H).

Ethyl N-(3,4-dichlorobenzyl)-5-methoxy-6-fluoroindole-2-carboxylate

NMR (CD₃SOCD₃) δ 1.25 (t, 3H), 3.8 (s, 3H), 4.2 (q, 2H), 5.75 (s, 2H), 6.9 (d, 1H), 7.3-7.4 (m, 3H), 7.5 (d, 1H), 7.6 (d, 1H)

Ethyl N-(3-trifluoromethyl-4-chlorobenzyl)-5-methoxy-6-fluoroindole-2-carboxylate

NMR (CD₃SOCD₃) δ 1.36 (t, 3H), 3.92 (s, 3H), 4.31 (q, 2H), 5.72 (s, 2H), 6.95-7.05 (m, 2H), 7.15 (d, 1H), 7.3 (s, 1H), 7.36 (d, 1H), 7.43 (s, 1H).

Ethyl N-(3,4-dichlorobenzyl-5-methoxy-4,6-dichloroindole-2-carboxylate

NMR (CDCl₃) δ 1.39 (t, 3H), 3.91 (s, 3H), 4.33 (q, 2H), 5.7 (s, 2H), 6.82 (dd, 1H), 7.11 (d, 1H), 7.24 (s, 1H), 7.34 (d, 1H), 7.42 (s, 1H)

Ethyl N-(3-trifluoromethyl-1-4dichlorobenzyl)-5methoxy-4,6-dichoroindole-2-carboxylate

NMR (CDCl₃) δ 1.4 (t, 3H), 3.95 (s, 3H), 4.35 (q, 2H), 5.75 (s, 2H), 7.09d, 1H), 7.25-7.5 (m, 4H).

Ethyl N-(3,4-dichlorobenzyl -5-methoxy-6-chloroindole-2-carboxylate

NMR (CDCl₃) δ 1.36 (t, 3H), 3.94 (s, 3H), 4.31 (q, 2H), 5.69 (s, 2H), 6.82 (dd, 1H), 7.09 (d, 1H), 7.14 (s, 1H), 7.24-7.35 (m, 3H); m/z 414 (MH⁺)

(iv) Ethyl N-(3-trifluoromethyl-4-chlorobenzyl)-5-hydroxy-6-chloroindole-2-carboxylate

A mixture of ethyl N-(3-trifluoromethyl-4-chlorobenzyl)-5-methoxy-6-chloroindole-2-§0 carboxylate (650 mgs) and trimethylsilyliodide (0.8 ml) in chloroform (50 ml) was stirred at 50° C. for 18 hours. Further aliquots of triethylsilyliodide were added until no starting material remained and the reaction was then poured into methanol (100 ml).The mixture was concentrated under vacuo and purified by column chromatography using 15% ethyl acetate/isohexane as the eluant to yield the desired product as a white solid (276 mg, 44%/O); NMR (CDCl₃) δ 1.36 (t, 3H), 4.31 (q, 2H), 5.75 (s, 2H), 7.0 (dd, 1H), 7.24-7.51 (m, 3H), 7.38 (d, 1H), 7.44 (d, 1H)

In a similar manner, but using ethyl N-(3-trifluoromethyl-4-fluorobenzyl)-5-methoxy-6-chloroindole-2-carboxylate or ethyl N-(3,4-dichlorobenzyl)-5-methoxy-6-fluoroindole-2-carboxylate or ethyl N-(3,4-dichlorobenzyl)-5-methoxy-4,6-dichloroindole-2-carboxylate or ethyl N-(3,4-dichlorobenzyl)-5-methoxy-6-chloroindole-2-carboxylate or ethyl N-(3-trifluoromethyl-4-chlorobenzyl)-5-methoxy-6-fluoroindole-2-carboxylate or ethyl N-(3-trifluoromethyl-4-dichlorobenzyl)-5-methoxy-4,6-dichloroindole-2-carboxylate were prepared:

Ethyl N-(3-trifluoromethyl-4-fluorobenzyl -5-hydroxy-6-chloroindole-2-carboxylate

53% yield. NMR (CD₃SOCD₃) δ 1.25 (t, 3H), 4.25 (q, 2H), 5.85 (s, 2H), 7.1-7.25 (m, 3H), 7.4 (t, 1H), 7.5 (d, 1H), 7.8 (s, 1H), 9.8 (s, 1H); m/z 414 (M−H⁺)

Ethyl N-(3.4-dichlorobenzyl -5-hydroxy-6-fluoroindole-2-carboxylate

31% yield. NMR (CDCl₃) δ 1.4 (t, 3H), 4.3 (q, 2H), 5.7 (s, 2H), 6.8 (dd, 1H), 7.0 (d, 1H), 7.1-7.3 (m, 2H); m/z 380 (M−H⁺)

Ethyl N-(3-trifluoromethyl-4-chlorobenzyl -5-hydroxy-6-fluoroindole-2-carboxylate

26% yield. NMR (CDCl₃) δ 1.35 (t, 3H), 4.31 (q, 2H), 4.98 (bd, 1H), 5.72 (s,2H), 6.96 (d, 1H), 7.01 (dd, 1H), 7.23-7.3 (m, 2H), 7.37 (d, 1H), 7.44 (s, 1H);m/z 414,416 (M−H⁺).

Ethyl N-(3,4-dichlorobenzyl -5-hydroxy-4,6-dichloroindole-2-carboxylate

69% yield. NMR (CDCl₃) δ 1.39 (t, 3H), 4.34 (q, 2H), 5.65 (bs, 1H), 7.7 (s, 2H), 6.82 (dd, 1H), 7.1 (d, 1H), 7.23 (s, 1H), 7.33 (d, 1H), 7.35 (s, 1H); m/z 436, 434, 432, 430 (M−H⁺)

Ethyl N-(3-trifluoromethyl-4-chlorobenzyl -5-hydroxy-46-dichloroindole-2-carboxylate

80% yield. NMR (CDCl₃) δ 1.39 (t, 3H), 4.36 (q, 2H), 5.66 (s, 1H), 5.75 (s, 2H), 7.0 (dd, 1H), 7.12 (s, 1H), 7.35-7.41 (m, 2H), 7.43 (d, 1H); 466, 468 (M−H⁺)

Ethyl N-(3,4-dichlorobenzyl -5-hydroxy-6-chloroindole-2-carboxylate

37% yield. m/z 398 (M−H⁺)

Method E2

Ethyl N-(3-trifluoromethyl-4-chlorobenzyl)-5-acetoxy-6-bromoindole-2-carboxylate

(i) Ethyl-5-methoxy-6-bromoindole-2-carboxylate

The procedure described in method E(i)-(ii) was repeated using 3-bromo-4methoxy aniline to give the desired product (24% yield): ¹H NMR (DMSO-d₆) 61.30 (t, 3H), 3.80 (s, 3H), 4.30 (q, 2H), 7.05 (m, 1H), 7.25 (s, 1H), 7.60 (s, 1H), 11.79 (s, 1H); m/z 296.3 (M−H⁻).

(ii) Ethyl N-(3-trifluoromethyl-4-chlorobenzyl -5-acetoxy-6-bromoindole-2-carboxylate

The procedure described in method A(i)-(iii) was repeated using the appropiate benzyl halide to give the desired product: ¹H NMR (DMSO-d₆) δ 1.22 (t, 3H), 2.32 (s, 3H), 4.25 (q, 2H), 5.90 (s, 2H), 7.10 (m, 1H), 7.40 (s, 1H), 7.60 (d, 1H), 7.63 (s, 1H), 7.68 (m, 1H), 8.10(s, 1H)

In a similar manner but using 3,4-dichlorobenzyl chloride was prepared:

Ethyl N-(3-trifluoromethyl-4-chlorobenzyl)-5-acetoxy-6-bromoindole-2-carboxylate m/z 486.2 (M−H⁺)

Method E3

Ethyl N-(3-trifluoromethyl-4-chlorobenzyl -5-hydroxy-7-fluoroindole-2-carboxylate (i) Ethyl N-(3-trifluoromethyl-4chlorobenzyl)-5-benzyloxy-7 fluoroindole-2-carboxylate

The procedure described in Method E(i)-(iii) was repeated using 2-fluoro-4-benzyloxy aniline as starting material to give the desired product (71% yield): ¹H NMR (DMSO-d₆) δ 1.22(t, 3H), 4.25(q, 2H), 5.10(s, 2H), 5.90(s, 2H), 6.95(m, 1H), 7.15(m, 2H), 7.30-7.50(m, 6H), 7.60(m, 2H)

(ii) Ethyl N-(3-trifluoromethyl-4-chlorobenzyl)-5-hydoxy-7 fluoroindole-2-carboxylate

To a solution of Ethyl N-(3-triuoromethyl-4-chlorobenzyl)-5-benzyloxy-7 fluoroindole-2-carboxylate (5omgs) in ethyl acetate (5 ml) was added a catalytic amount of 5% palladium on carbon and the resulting was stirred under a hydogen atnmosphere for 72 hours. The mixture was filtered and concentrated in vacuo to give the desired product (59 mg): m/z 414.25 (M−H⁺)

### EXAMPLE 27

Pharmaceutical Compositions

This Example illustrates, but is not intended to limit, representative pharmaceutical dosage forms of the invention as defined herein (the active ingredient being termed “Compound X”), for therapeutic or prophylactic use in humans:

| (a) | |
|---|---|
| Tablet I | mg/tablet |
| Compound X. | 100 |
| Lactose Ph.Eur | 182.75 |
| Croscarmellose sodium | 12.0 |
| Maize starch paste (5% w/v paste) | 2.25 |
| Magnesium stearate | 3.0 |

| (b) | |
|---|---|
| Tablet II | mg/tablet |
| Compound X | 50 |
| Lactose Ph.Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |

| (c) | |
|---|---|
| Tablet III | mg/tablet |
| Compound X | 1.0 |
| Lactose Ph.Eur | 93.25 |
| Croscarmellose sodium | 4.0 |
| Maize starch paste (5% w/v paste) | 0.75 |
| Magnesium stearate | 1.0 |

| (d) | |
|---|---|
| Capsule | mg/capsule |
| Compound X | 10 |
| Lactose Ph.Eur | 488.5 |
| Magnesium | 1.5 |

| (e) | |
|---|---|
| Injection I | (50 mg/ml) |
| Compound X | 5.0% w/v |
| 1 M Sodium hydroxide solution | 15.0% v/v |
| 0.1 M Hydrochloric acid | to adjust pH to 7.6 |
| Polyethylene glycol 400 | 4.5% w/v |
| Water for injection | to 100% |

| (f) | |
|---|---|
| Injection II | (10 mg/ml) |
| Compound X | 1.0% w/v |
| Sodium phosphate BP | 3.6% w/v |
| 0.1 M Sodium hydroxide solution | 15.0% v/v |
| Water for injection | to 100% |

| (g) | |
|---|---|
| Injection III | (1 mg/ml, buffered to pH6) |
| Compound X | 0.1% w/v |
| Sodium phosphate BP | 2.26% w/v |
| Citric acid | 0.38% w/v |
| Polyethylene glycol 400 | 3.5% w/v |
| Water for injection | to 100% |

| (h) | |
|---|---|
| Aerosol I | mg/ml |
| Compound X | 10.0 |
| Sorbitan trioleate | 13.5 |
| Trichlorofluoromethane | 910.0 |
| Dichlorodifluoromethane | 490.0 |

| (i) | |
|---|---|
| Aerosol II | mg/ml |
| Compound X | 0.2 |
| Sorbitan trioleate | 0.27 |
| Trichlorofluoromethane | 70.0 |
| Dichlorodifluoromethane | 280.0 |
| Dichlorotetrafluoroethane | 1094.0 |

| (j) | |
|---|---|
| Aerosol III | mg/ml |
| Compound X | 2.5 |
| Sorbitan trioleate | 3.38 |
| Trichlorofluoromethane | 67.5 |
| Dichlorodifluoromethane | 1086.0 |
| Dichlorotetrafluoroethane | 191.6 |

| (k) | |
|---|---|
| Aerosol IV | mg/ml |
| Compound X | 2.5 |
| Soya lecithin | 2.7 |
| Trichlorofluoromethane | 67.5 |
| Dichlorodifluoromethane | 1086.0 |
| Dichlorotetrafluoroethane | 191.6 |

| (l) | | |
|---|---|---|
| Ointment | ml | |
| Compound X | 40 | mg |
| Ethanol | 300 | μl |
| Water | 300 | μl |
| 1-Dodecylazacycloheptan-2-one | 50 | μl |

| | |
|---|---|
| Propylene glycol | to 1 ml |

Note:
Compound X in the above formulations may comprise a compound as illustrated in Examples herein.

The above formulations may be obtained by conventional procedures well known in the pharmaceutical art. The tablets (a)-(c) may be enteric coated by conventional means, for example to provide a coating of cellulose acetate phthalate. The aerosol formulations (h)-(k) may be used in conjunction with standard, metered dose aerosol dispensers, and the suspending agents sorbitan trioleate and soya lecithin may be replaced by an alternative suspending agent such as sorbitan monooleate, sorbitan sesquioleate, polysorbate 80, polyglycerol oleate or oleic acid

1. A compound of the formula (I): 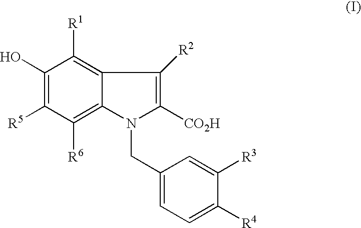
wherein:
R¹ is hydrogen, halo or methoxy,
R² is hydrogen, halo, methyl, ethyl or methoxy;
R³ is a halo group or a trifluoromethyl group;
R⁴ is a halo group or a trifluoromethyl group;
R⁵ is hydrogen or halo;
R⁶ is hydrogen or halo;
provided that when R⁵ and R⁶ are both hydrogen, and one of R³ or R⁴ is chloro or fluoro, then the other is not chloro or fluoro;
or a pharmaceutically acceptable salt or prodrug thereof.

2. A compound according to claim 1, wherein in the formula (I):
R¹ is hydrogen, fluoro or chloro;
R² is hydrogen, chloro, bromo, iodo or methoxy;
R³ is fluoro, chloro, bromo, iodo;
R⁴ is trifluoromethyl;
R⁵ and R⁶ are hydrogen.

3. A compound according to claim 1, wherein in the formula (I):
R¹ is hydrogen, fluoro or chloro;
R² is hydrogen, chloro, bromo, iodo or methoxy;
R³ is trifluoromethyl;
R⁴ is fluoro, chloro, bromo or iodo;
R⁵ and R⁶ are hydrogen.

4. A compound according to claim 1, wherein in the formula (I):
R¹ is hydrogen, fluoro or chloro;
R² is hydrogen, chloro, bromo, iodo or methoxy,
R³ and R⁴ are both halo, especially fluoro, chloro or bromo, or one of R³ and R⁴ is chloro and the other one of R³ and R⁴ is fluoro;
one or both of R⁵ and R⁶ is halo.

5. A compound according to claim 1 which is a compound of formula (IA): 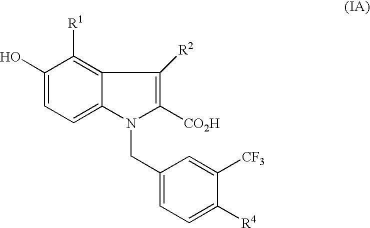
wherein R¹, R² and R⁴ are as defined in claim 1, or a pharmaceutically acceptable salt or prodrug thereof.

6. A compound according to claim 1 which is any of the following:
N-(3-trifluoromethyl-4-chlorobenzyl)-5-hydroxyindole-2-carboxylic acid;
N-(3-fluoro-4-trifluoromethylbenzyl)-5-hydroxyindole-2-carboxylic acid;
N-(3-chloro-4trifluoromethylbenzyl)-5-hydroxyindole-2-carboxylic acid;
N-(3-bromo-4-chlorobenzyl)-5-hydroxyindole-2-carboxylic acid;
N-(3-fluoro-4-bromobenzyl)-5-hydroxyindole-2-carboxylic acid;
N-(3-bromo-4-fluorobenzyl)-5-hydroxyindole-2-carboxylic acid;
N-(3-trifluoromethyl-4-fluorobenzyl)-4-fluoro-5-hydroxyindole-2-carboxylic acid
N-(3-trifluoromethyl-4-chlorobenzyl)-4-fluoro-5-hydroxyindole-2-carboxylic acid;
N-(3-trifluoromethyl-4-fluorobenzyl)-4,6-difluoro-5-hydroxyindole-2-carboxylic acid;
N-(3,4-chlorobenzyl)-4,6-dichloro-5-hydroxyindole-2-carboxylic acid;
N-(3-trifluoromethyl-4-fluorobenzyl)-3-bromo-5-hydroxyindole-2-carboxylic acid;
N-(3-trifluoromethyl-4chlorobenzyl)-3-bromo-5-hydroxyindole-2-carboxylic acid;
N-(3-chloro-4-trifluoromethylbenzyl)-3-bromo-5-hydroxyindole-2-carboxylic acid;
N-(3-fluoro-4-trifluoromethylbenzyl)-3-chloro-5-hydroxyindole-2-carboxylic acid;
N-(3-fluoro-4-trifluoromethylbenzyl)-3-iodo-5-hydroxyindole-2-carboxylic acid;
N-(3-trifluoromethyl-4-chlorobenzyl)-3-methoxy-5-hydroxyindole-2-carboxylic acid;
N-(3-trifluoromethyl-4-fluorobenzyl)-5-hydroxy-6-chloroindole-2-carboxylic acid;
N-(3-trifluoromethyl-4-chlorobenzyl)-5-hydroxy-6-chloroindole-2-carboxylic acid;
N-(3-trifluoromethyl-4-chlorobenzyl)-5-hydroxy-7-fluoroindole-2-carboxylic acid;
N-(3-trifluoromethyl-4-chlorobenzyl)-5-hydroxy-6-bromoindole-2-carboxylic acid;
N-(3,4-dichlorobenzyl)-5-hydroxy-6-bromoindole-2-carboxylic acid;
N-(3-trifluoromethyl-4-chlorobenzyl)-5-hydroxy-6-fluoroindole-2-carboxylic acid;
N-(3,4-dichlorobenzyl)-5-hydroxy-6-fluoroindole-2-carboxylic acid;
N-(3,4-dichlorobenzyl)-5-hydroxy-6-chloroindole-2-carboxylic acid;
N-(3-trifluoromethyl-4chlorobenzyl)-4-chloro-5-hydroxyindole-2-carboxylic acid;
N-(3-trifluoromethyl-4-chlorobenzyl)4,6-dichloro-5-hydroxyindole-2-carboxylic acid;
N-(3-trifluoromethyl-4-chlorobenzyl)-5-acetoxyindole-2-carboxylic acid (prodrug of N-(3-trifluoromethyl-4chlorobenzyl)-5-hydroxyindole-2-carboxylic acid).

7. A process for preparing a compound according to claim 1 or a pharmaceutically acceptablehsalt or prodrug thereof, which process comprises:
(a) reacting a compound of formula (II): 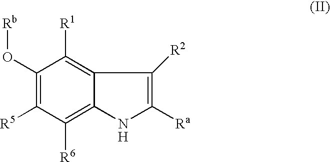
where R¹, R², R⁵ and R⁶ are as defined in claim 1, R^{a} is carboxy or a protected form thereof, and R^{b} is hydrogen or a suitable hydroxy protecting group, with a compound of formula (III): 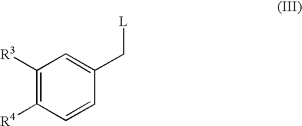
where R³ and R⁴ are as defined in claim 1 and L is a displaceable group; and optionally thereafter:
(b) (i) converting a resulting compound of the formula (I) into another compound of the formula (I);
(ii) removing any protecting groups; or
(iii) forming a pharmaceutically acceptable salt or prodrug thereof.

8. A compound according to any one of claims 1 to 6 or a pharmaceutically acceptable salt or prodrug thereof, for use in a method of treatment of the human or animal body by therapy.

9. A compound according to any one of claims 1 to 6 or a pharmaceutically acceptable salt or prodrug thereof, for use as a medicament.

10. A compound according to claim 9, which is for use as a medicament for antagonising an MCP-1 mediated effect in a warm-blooded animal.

11. A pharmaceutical composition comprising a compound according to any one of claims 1 to 6 or a pharmaceutically acceptable salt or prodmg thereof, in association with a pharmaceutically acceptable excipient or carrier.

12. Use of a compound according to any one of claims 1 to 6 or a pharmaceutically acceptable salt or prodrug thereof, in the manufacture of a medicament for use in antagonising an MCP-1 mediated effect in a warm-blooded animal.

13. A method of treating inflammatory disease which comprises administering to a host in need of such treatment a compound according to any one claims 1 to 6 or a pharmaceutically acceptable salt or prodrug thereof, or a pharmaceutical composition according to claim 11.

14. A method of antagonising an MCP-1 mediated effect in a warm-blooded animal in need of such treatment which comprises administering to said animal an effective amount of a compound according to any one of claims 1 to 6 or a pharmaceutically acceptable salt or prodrug thereof, or a pharmaceutical composition according to claim 11.
